(19) United States
(12) Patent Application Publication
(10) Pub. No.: US 2006/0182805 A1
(43) Pub. Date: Aug. 17, 2006

(54) Dosage form and method for sustained release of substituted pyrazine compound

(75) Inventors: James Pfeiffer, Oakland, CA (US); Mark Foreman, San Mateo, CA (US); Michael Des Jardin, Sunnyvale, CA (US); Janne Wissel, Sunnyvale, CA (US); Samuel Saks, Burlingame, CA (US)

Correspondence Address: JAZZ PHARMACEUTICALS, 318 PORTER DRIVE, PALO ALTO, CA, 94304, US

(73) Assignee: Jazz Pharmaceuticals, Palo Alto, CA (US)

(21) Appl. No.: 11/353,572

(22) Filed: Feb. 14, 2006

Related U.S. Application Data
(60) Provisional application No. 60/653,444, filed on Feb. 15, 2005.

(51) Int. Cl. A61K 31/4965; A61K 9/22; A61K 9/24

(52) U.S. Cl. 424/468; 424/473; 514/255.06

(57) ABSTRACT

The invention is directed to a dosage form and method for administering a therapeutic agent in a sustained release manner to provide an intended therapeutic effect while minimizing the side effects associated with the therapeutic agent. The therapeutic agent is selected from a group of substituted pyrazine compounds and may be 3-(2,3,5-trichloro-phenyl)-pyrazine-2,6-diamine.

This application claims priority from Provisional Application No. 60/653,444 filed Feb. 15, 2005 which is incorporated herein by reference.

### BACKGROUND OF THE INVENTION

This invention relates to a method and dosage form for delivery of pyrazine derivatives. The compounds are released from the dosage form in a fashion that permits once daily dosing. The invention also relates to methods of treating psychiatric disorders and disorders of the central nervous system.

U.S. Pat. No. 6,255,307 describes a series of pyridine derivatives that are sodium channel blockers and useful as anti-convulsants, mood stabilizers and analgesics. It is noted in the patent that the compounds are particularly useful for treating epilepsy, bipolar disorder, as analgesics for treating or preventing pain, for treatment of functional bowel disorders, for the treatment of neurodegenerative diseases and for preventing or reducing dependence on, or preventing or reducing tolerance or reverse tolerance to a dependence-inducing agent. The compounds may be formulated into a conventional dosage form suitable for oral, parenteral, rectal or topical administration.

The above-noted patent describes delivery of the compounds by conventional, immediate release dosage forms. The peak and trough phenomena produced by immediate release dosage forms is a drawback, as such a delivery profile may result in a peak concentration that is higher than therapeutically necessary and a trough concentration that is lower than necessary to provide a therapeutic benefit. Moreover, the peak and trough delivery pattern provided by known dosage forms may result in undesirable variation in the therapeutic effect. Another undesirable aspect of conventional immediate release dosage forms for some diseases is the need to increase the dose in a step-wise fashion periodically in order to determine the therapeutically effective dose. The step-wise increase may produce even greater fluctuations in the plasma level as steady state is attained at the higher dose. Conventional dosage forms may also require several doses before attaining steady state plasma levels. This increases the number of doses before a therapeutically efficacious dose is reached. In addition to undesirable variation in therapeutic effect of such plasma level fluctuations, an increase in undesirable side effects may result.

It would therefore be useful to provide a dosage form for the above described compounds that would allow for controlled-release delivery of the compounds at a therapeutically effective concentration range for an extended period of time. Such delivery would reduce the side-effects associated with the peak plasma concentration and lack of efficacy at the trough concentration levels of conventional dosage forms and allow for treatment or prevention of disease states for which the side effects would be unacceptable.

### BRIEF SUMMARY OF THE INVENTION

In one aspect, the invention is a sustained release dosage form comprising a substitute pyrazine derivative where the plasma concentration range at steady state remains constant such that Cmax over Cmin at steady state is less than or equal to 3.

In another aspect, the invention is a sustained release dosage form comprising a substituted pyrazine derivative wherein the dosage form is effective to provide sustained release over a period of at least about 8 hours.

In one embodiment, the dosage form is effective to provide a zero-order dissolution rate over a period of 8 to 24 hours and in another embodiment the dosage form is effective to provide a first-order dissolution rate over a period of 8 to 24 hours.

Such a dosage form would have a dissolution rate where between 2% and 50% of the dose is released following two hours after exposure to an aqueous environment. The dosage form may be an osmotic dosage form, matrix dosage form, multilayer dosage form, or it may be in a coated bead form.

In another aspect, the invention is a method for treating a central nervous system disorder comprising administering to a human subject a sustained release dosage form of a substituted pyrazine derivative. In a specific embodiment, the disorder may include epilepsy. In further embodiments, these disorders may include drug induced or naturally occurring dyskinesias, off-phase tachyphylaxis to L-DOPA treatment in Parkinson's, movement disorders, dementias, cognitive disorders associated with neurodegenerative disorders, stroke, fibromyalgia, acute and chronic pain, migraine and pseudobulbar affect.

In still another aspect, the invention is a method for treating psychiatric disorders comprising administering to a human subject a sustained release dosage form of a substituted pyrazine derivative. In specific embodiments, these disorders may include unipolar depression, bipolar depression, major depressive disorder, positive and negative symptoms of schizophrenia, post traumatic stress syndrome, acute mania, panic and psychotic reactions and attacks, conduct disorders, intermittent explosive or antisocial disruptive disorders, anxiety disorders. Cognitive symptoms of schizophrenia, borderline personality disorder, attention deficit disorder, alcoholism and substance abuse.

In a further aspect, the invention is a sustained release dosage form comprising 3-(2,3,5-trichloro-phenyl)-pyrazine-2,6-diamine where the plasma concentration range at steady state remains constant such that the Cmax to Cmin ratio at steady state is less than or equal to 3.

In another aspect, the invention is a sustained release dosage form comprising 3-(2,3,5-trichloro-phenyl)-pyrazine-2,6-diamine wherein the dosage form is effective to provide sustained release over a period of at least about 8 hours.

In another aspect, the invention is a sustained release dosage from comprising 3-(2,3,5-trichloro-phenyl)-pyrazine-2,6-diamine wherein the dosage form is effective to provide sustained release over a period of at least about 14 to 24 hours.

In another aspect, the invention is a method for treating a central nervous system disorder comprising administering to a human subject a sustained release dosage form of 3-(2,3,5-trichloro-phenyl)-pyrazine-2,6-diamine. In a specific embodiment the disorder may include epilepsy. In further embodiments, these disorders may include drug induced or naturally occurring dyskinesias, off-phase tachyphylaxis to L-DOPA treatment in Parkinson's, movement disorders, dementias or cognitive disorders associated with neurodegenerative disorders, stroke, fibromyalgia, acute and chronic pain, migraine and pseudobulbar affect.

In still another aspect, the invention is a method for treating psychiatric disorders comprising administering to a human subject a sustained release dosage form of 3-(2,3,5-trichloro-phenyl)-pyrazine-2,6-diamine. In specific embodiments, these disorders may include unipolar depression, major depressive disorder, positive and negative symptoms of schizophrenia, post traumatic stress syndrome, acute mania, panic and psychotic reactions and attacks, conduct disorders, disorders, intermittent explosive or antisocial disruptive disorders, anxiety disorders, cognitive symptoms of schizophrenia, borderline personality disorder, attention deficit disorder, alcoholism and substance abuse.

In yet another aspect, the invention is a method for maintaining the therapeutic effect of 3-(2,3,5-trichloro-phenyl)-pyrazine-2,6-diamine by administering to a human subject a sustained release dosage form comprising 3-(2,3,5-trichloro-phenyl)-pyrazine-2,6-diamine that delivers the 3-(2,3,5-trichloro-phenyl)-pyrazine-2,6-diamine in a controlled and increasing dose over about 12 to 24 hours to achieve the therapeutic effect in the subject. It may be possible to use a combination of immediate release dosage forms and/or sustained release dosage forms to titrate and maintain therapeutic levels.

### BRIEF DESCRIPTION OF THE DRAWINGS

FIG. 1 shows the simulated PK profile of a single dose of 3-(2,3,5-trichloro-phenyl)-pyrazine-2,6-diamine delivered orally from a conventional dosage form.

FIG. 2 shows the simulated PK profiles of a single dose of a sustained release dosage form of 3-(2,3,5-trichloro-phenyl)-pyrazine-2,6-diamine in vivo and in vitro.

FIG. 3 shows the release rates of 3-(2,3,5-trichloro-phenyl)-pyrazine-2,6-diamine from a dosage form according to the invention in a zero-order release profile.

FIG. 4 shows the release rates of 3-(2,3,5-trichloro-phenyl)-pyrazine-2,6-diamine from a further dosage form according to the invention in a first order release profile.

FIG. 5 shows the in vitro release profile of a single from a further dosage form according to the invention that delivers 3-(2,3,5-trichloro-phenyl)-pyrazine-2,6-diamine in a controlled and increasing rate.

FIG. 6 shows the simulated steady state plasma profile resulting from delivery of a dosage form in a zero-order profile after administration of the first dose and multiple doses thereafter until steady state is achieved.

FIG. 7 shows the structure of 3-(2,3,5-trichloro-phenyl)-pyrazine-2,6-diamine and its metabolites M1, M2, M3, M4 and M5.

FIG. 8 shows the cumulative release rates of 3-(2,3,5-trichloro-phenyl)-pyrazine-2,6-diamine from a 50 mg osmotic dosage form in 3 different membrane weights according to the invention.

FIG. 9 is a simulation of the plasma concentration of the 25 mg membrane dosage form described in Example 5 and with the release rate shown in FIG. 8.

FIG. 10 shows the release profile of 3-(2,3,5-trichloro-phenyl)-pyrazine-2,6-diamine from a coated bead dosage form according to Example 6.

FIG. 11 is a simulation of the plasma concentration of the dosage form described in Example 6 with the release rate shown in FIG. 10.

### DETAILED DESCRIPTION OF THE INVENTION

### DEFINITIONS

By “substituted pyridine derivative” is intended the compounds described in U.S. Pat. No. 6,255,307 and having the following structure:

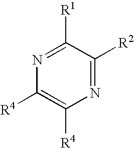

wherein

R¹ is selected from the group consisting of phenyl substituted by one or more halogen atoms, naphthyl and naphthyl substituted by one or more halogen atoms;

R² is selected from the group consisting of —NH₂ and —NHC(═O)R^{a};

R³ is selected from the group consisting of NR^{b}R^{c}, —NHC(═O)R^{a} and hydrogen;

R⁴ is selected from the group consisting of hydrogen, —C₁₋₄ alkyl, —C₁₋₄ alkyl substituted by one or more halogen atoms, —CN, —CH₂OH, —CH₂OR^{d} and —CH₂S(O)ₓR^{d};

wherein

R^{a} represents C₁₋₄ alkyl or C₃₋₇ cycloalkyl, and

R^{b} and R^{c}, which may be the same or different, are selected from hydrogen and C₁₋₄ alkyl, or together with the nitrogen atom to which they are attached, form a 6-membered nitrogen containing heterocycle, which heterocycle can be further substituted with one or more C₁₋₄ alkyl;

R^{d} is selected from C₁₋₄ alkyl or C₁₋₄ alkyl substituted by one or more halogen atoms;

x is an integer zero, one or two;

and pharmaceutically acceptable derivatives thereof;

with the proviso that R¹ does not represent:

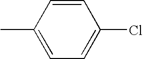

when R² is —NH₂, and both R3 and R4 are hydrogen.

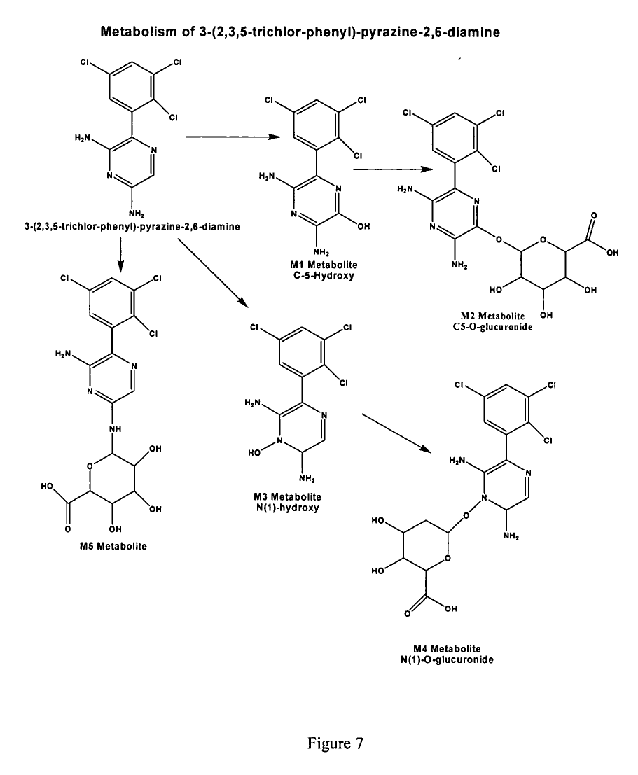

Pyrazine derivatives also include various forms of such compounds including but not limited to uncharged molecules, components of molecular complexes or nonirritating, pharmacologically acceptable salts. Also, simple derivatives of the agents such as ethers, esters, amides, etc., which are easily hydrolyzed by body pH, enzymes etc. can be employed. Further, the metabolites shown in FIG. 7, denoted the M1, M2, M3, M4 and M5 metabolites may be included as the active agents in the dosage form of the invention.

By “3-(2,3,5-trichloro-phenyl)-pyrazine-2,6-diamine” in intended the compound with the following structure:

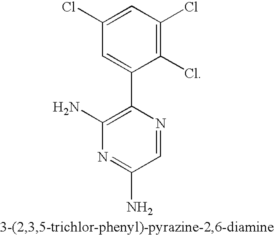

By “osmotic dosage form” is intended a dosage form with a semipermeable wall that surrounds a therapeutic composition. In use within a patient, the osmotic dosage form imbibes fluid through the semipermeable wall into the dosage form in response to the concentration gradient across the semipermeable wall. The therapeutic composition in the dosage form develops osmotic energy that causes the therapeutic composition to be administered through an exit from the dosage form over a prolonged period of time up to 24 hours (or even in some cases up to 30 hours) to provide controlled and sustained therapy. In one embodiment, the dosage form comprises a wall surrounding a compartment, the wall comprising a semipermeable polymeric composition permeable to the passage of fluid and substantially impermeable to the passage of therapeutic agent present in the compartment, a therapeutic agent layer composition in the compartment comprising the therapeutic agent; a hydrogel push layer composition in the compartment comprising an osmotic formulation for imbibing and absorbing fluid for expanding in size for pushing the therapeutic agent composition layer from the dosage form; and at least on passageway in the wall for releasing the therapeutic agent composition. It is possible that dosage forms of this type may have a push layer that is a non-hydrogel push layer or that may not require a separate push layer and the osmotic driving force can be incorporated into a single layer.

By “matrix dosage form” is intended a dosage form in which the therapeutic agent is admixed with at lease one hydrophilic polymer, but may contain two or more hydrophilic polymers. When hydrated, the polymer forms a gel layer around the dry tablet core. The matrix dosage form is made of low or high viscosity erodible polymers or mixtures thereof. The polymers may be hydrophilic cellulose derivatives and may be granulated together with the therapeutic agent and optionally lubricants, glidants and other additives prior to being compressed into tablets for oral delivery.

By “coated bead dosage form” is intended a dosage form with one or more units having the same or varying concentrations of therapeutic agent, designed to release its contents at varying times. In one embodiment, the dosage form comprises three different types of beads in a single multiple-unit dosage form. The first unit is an immediate release dosage unit in bead form. The bead may have a surface-active agent such as sodium laurel sulfate, sodium monoglycerate, sorbitan monooleate, any one of the pluronic line of surface active polymers or combinations of any of the above in combination with the therapeutic agent. The first bead should release the therapeutic agent within 6 hours, often within 4 hours or 2 hours or 1 hour after delivery. The second and third units are extended release dosage units in bead form. In addition to the ingredients of the first unit, the second bead should have a controlling coat applied to the surface of the bead such that the release of the bead is controlled and released over a period of from about 2 to 14, often 4 to 12 hours after delivery. This second unit should release its contents in the duodenum, ileum or jejunum. The third unit is coated such that it will release its contents in the colon. Such bead should release the therapeutic agent over a period of from about 10 to 24 hours, often 12 to about 20 hours after delivery. For each type of bead composition, the coated layer can contain various polymer ingredients that optionally may have various dissolution profiles as a function of pH or other physiological condition.

By “multilayer dosage form” is intended a dosage form with a multiple layers (2-6 layers), each layer containing therapeutic agent. The layers are laminated one to the other with the outermost layer surface coated with a drug impermeable polymer, such that the therapeutic agent is release first from the outermost layer. These biodegradable polymer layers undergo sequential chemical decomposition to form soluble monomers or polymer units. Representative polymers include: polyamides, polyamino acid), polyesters, polylactic acid, polyglycolic acid, polyothoesters, and polyanhydrides.

By “dissolution rate” or “release rate” is intended the amount of active agent released in vitro from a dosage form per unit time into a release medium, usually in units of milligrams per hour (mg/hr). In vitro release rates are performed on dosage forms placed in metal coil sample holders attached to a USP Type VII bath indexer in a constant temperature water bath at 37° C. In addition, other appropriate dissolution test apparatuses may be suitable, such as, USP apparatus I or II. Aliquots of the release rate solutions are injected into a chromatographic system to quantify the amounts of drug released during each testing interval. According to the invention, between 2 and 50% of the dose is released following two hours after exposure of the dosage form to the environment of use. These dissolution rates may be between 2 and 60% or 10 and 50% within the first 4 hours, between about 20 and 80% or 35 and 70% from 4 hours to about 8 hours following exposure to the environment of use between about 35 and 95% or 45 and 85% from 8 hours to about 12 hours following exposure to the environment of use and greater than 50 to 80% or greater than 90% following 12 hours after exposure to the environment of use and greater than 90% following 22 hours after exposure to the environment of use.

By “plasma profile” or “plasma drug concentration” or “C” is intended the concentration of drug in the plasma of a subject, generally expressed as mass per unit volume, typically nanograms per milliliter (ng/ml).

By “zero-order” is intended a constant, linear, continuous, sustained and controlled release rate of therapeutic agent from a dosage form, the plot of mass of therapeutic agent released vs. time is linear for the majority of the release interval.

By “first-order” is intended a continuous, monotonically decreasing, sustained and controlled release rate of therapeutic agent from the dosage form, the plot of mass of therapeutic agent vs. time is log-linear.

By “increasing” is intended a continuous, increasing sustained and controlled release rate of the therapeutic agent from the dosage form, the plot of which may be represented by two or more linear release patterns.

By “therapeutically effective amount” is intended the amount of therapeutic agent necessary to effect the desired pharmacologic, often beneficial result. In practice, this will vary widely depending upon the severity of the condition and the desired therapeutic effect, but in general the amount will be between 0.1 mg and 1000 mg of therapeutic agent, often between about 5 and 400 mg, or between 25 and 200 mg.

By “extended release” is intended a continuous release of the therapeutic agent over an extended period of time of about 8 hours, 10 hours, 12 hours, 14 hours or of about 16 hours, or of about 20, 24 or 30 hours or of between 2 and 14 hours, or between 2 and 20 hours and often of between 4 and 16 hours or between 8 and 24 hours.

By “Cmax” is intended the maximum concentration of drug in the plasma of a subject, generally expressed as mass per unit volume, typically 10 to 1200 and often 100 to 1000 or 200 to 800 nanograms per milliliter (ng/ml)

By “Cmin” is intended the minimum concentration of drug in the plasma of a subject at steady state, generally expressed as mass per unit volume, typically 1 to 400 and often 30 to 300 or 50 to 200 nanograms per milliliter (ng/ml).

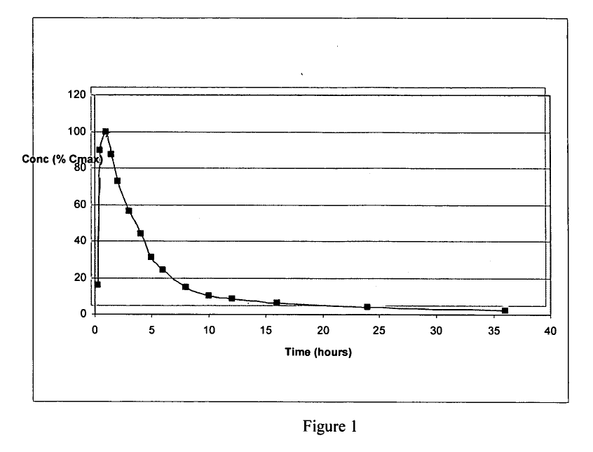

We have now found that an oral dosage form can be provided that administers a therapeutic agent in a sustained release fashion that may eliminate unwanted side effects. It may also allow for faster “titration” to a therapeutic dose. FIG. 1 shows the delivery of 3-(2,3,5-trichloro-phenyl)-pyrazine-2,6-diamine from an immediate release tablet. As can be observed, the initial rise of the drug is dramatic and occurs within 2 hours of oral delivery. Such peak concentrations often lead to unwanted side effects that include dizziness, ataxia, diplopia and rash. The concentration of therapeutic agent in the plasma after 4 hours is less than 50% of the maximum concentration. Such decrease in concentration may indicate that delivery is no longer within the minimum effective concentration range.

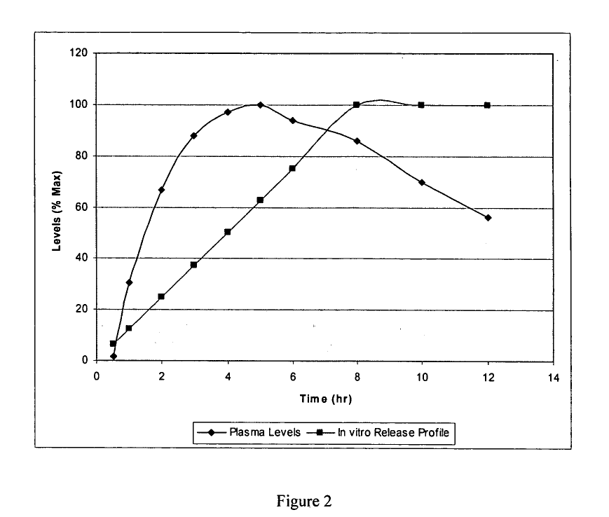

FIG. 2 shows the in vitro and in vivo profiles of a dosage form according to one embodiment of the invention. The in vitro release profile shows the dissolution rate of the dosage form containing 3-(2,3,5-trichloro-phenyl)-pyrazine-2,6-diamine according to the invention. Dissolution occurs in a zero-order fashion over a period of 8 hours. The plasma levels indicate that maximum plasma concentration occurs at about 6 hours following delivery and levels above 50% of maximum can be maintained using the dosage form of the invention for a period of at least about 10 hours. The result shows that the plasma levels can be maintained above about 50% of maximum for a period longer than the time that the drug is released by the dosage form. Such dosage forms allow for decreased interpatient and intrapatient variability, that is, reduced variability in plasma concentrations between different patients as well as decreased variability in plasma concentrations in an individual patient from day to day.

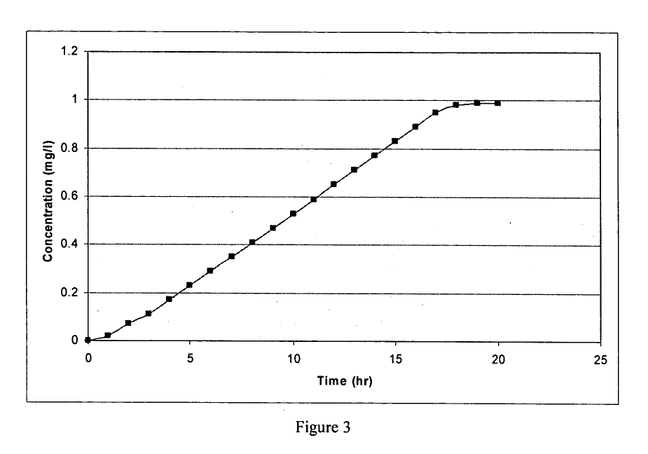

FIG. 3 shows a zero-order release profile according another embodiment of the invention. Release of active agent occurs over a period of about 20 hours with 90% released after 16 hours.

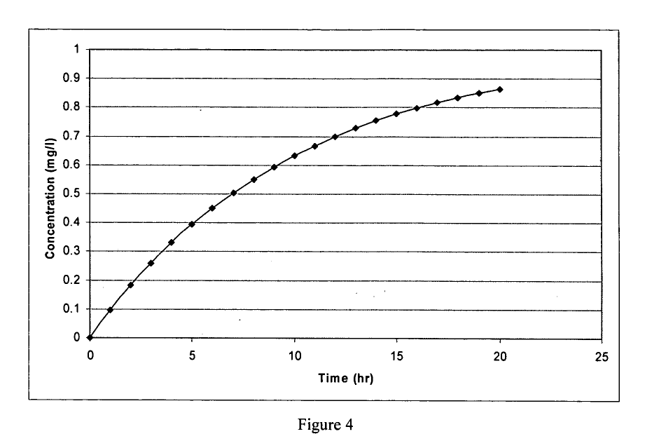

FIG. 4 shows the first-order release profile according to another embodiment of the invention. Release of the active agent occurs over a period of about 20 hours with 90% released after 11 hours. This release rate may be achieved by using an initial loading dose followed by a sustained release dose.

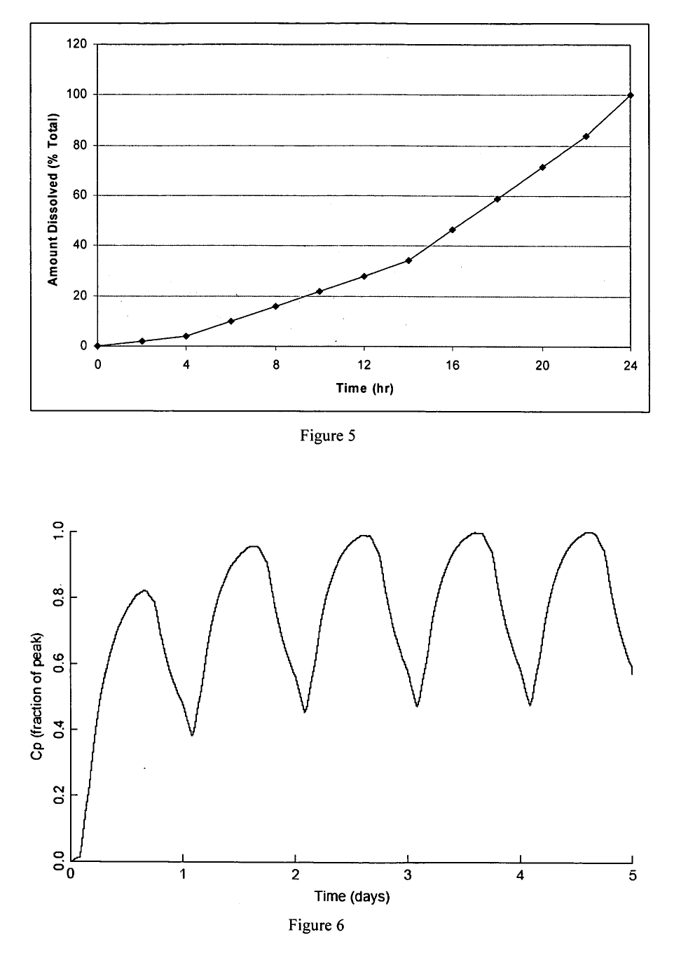

FIG. 5 shows the ascending release profile according to another embodiment of the invention that releases the drug in a controlled and increasing dose over 24 hours. This type of release pattern may be particularly advantageous for the dosage form containing 3-(2,3,5-trichloro-phenyl)-pyrazine-2,6-diamine which has a volume of distribution (Vd) of about 640 liters and shows possible multi-exponential elimination after single oral dose. The control of plasma levels is especially difficult when the compound shows such multi-exponential elimination. In this case, the drug is eliminated rapidly from the plasma via distribution into other tissues as well as through metabolism and excretion into the urine. The rapid elimination makes it necessary to deliver the drug into the GI tract for extended periods of time, up to about 24 hours. It may also require that the drug be delivered in an ascending profile such as that shown in this FIG. 5. Such ascending profile may be accomplished by increasing the dose from an initial dose in two to five increments to achieve a desired maximum dose or by delivering a dosage form with an ascending profile for the initial two to five doses. Such doses may be delivered every 24 hours or every 12 hours.

FIG. 6 shows the plasma profile resulting from the delivery of a dosage form according to the invention once per day for a period of 5 days. A steady state plasma profile occurs after the administration of two to three doses. The achievement of steady state with the desirable Cmax/Cmin ratio after the administration of very few doses is advantageous in that therapeutic levels are achieved in a short time and maintained thereafter. Rapid attainment of steady state allows for faster increases or decreases in dose without unwanted side effects and the resultant titration to a therapeutically effective dose and, if necessary, withdrawal of therapy. The Cmax/Cmin ratio at steady state is less than 3, often less than 2, often less than 1.5 and between 1.0 and 2. The initial half life of the drug is less than 5 hours, the final half life of the drug is between about 10 and 13 hours and the release period is at least about 20 hours.

The dosage forms of the present invention may be useful to treat a variety of conditions. These include CNS disorders of epilepsy, pain (acute pain such as muscoskeletal pain, post operative pain and surgical pain, chronic pain such as chronic inflammatory pain (i.e. rheumatoid arthritis and osteoarthritis) neuropathic pain (i.e. post herpetic neuralgia, trigeminal neuralgia, sympathetically maintained pain and pain associated with diabetic neuropathy) and pain associated with cancer and fibromyalgia or pain associated with migraine), oedema, multiple sclerosis, fibromyalgia, pseudobulbar affect, drug induced or naturally occurring dyskinesias, off-phase tachyphylaxis to L-DOPA treatment in Parkinson's, movement disorders, dementias or cognitive disorders including those associated with neurodegenerative disorders and stroke.

The dosage form may also be useful for treating psychiatric conditions including bipolar disorder, schizophrenia, bipolar depressive disorder, post-traumatic stress disorder, unipolar depression, major depressive disorder, positive and negative symptoms of schizophrenia, cognitive symptoms of schizophrenia, acute mania, panic and psychotic reactions and attacks, conduct disorders, intermittent explosive or disruptive disorders, anxiety disorders, traumatic stress syndrome, borderline personality disorder, attention deficit disorder, alcoholism and substance abuse. The dosage forms may also be useful in improving cognition.

The therapeutic agent may be delivered alone or in combination with other active agents. These active agents may be incorporated within the same dosage form of may be given to the patient in a separate dosage form. These active agents are selected from the group consisting of carbamazepine, oxcarbazepine, sodium valproate, gabapentin, vigabatrin, diazepam, L-DOPA, lithium, antidepressants which include but are not limited to compounds which possess activities such as serotonin reuptake inhibitors (SSRI's) such as citalopram, fluoxetine, paroxetine and sertraline and their pure enantiomers, norepinephrine reuptake (NERI) such as atomoxetine; combined serotonin and norepinephrine reuptake inhibitors (SNERI's) such as venlafaxine and duloxetine; dopamine reuptake inhibitors such as bupropion and compounds which possess multiple reuptake inhibition activities for serotonin, norepinephrine and/or dopamine; monoamine oxidase inhibitors such as phenelzine and tranylcypromine; agonists for the 5-HT1a receptor such as buspirone; 5-HT2 antagonists such as mirtazepine, anxiolytics including but not limited to benzodiazepines such as diazepam, alprazolam and clonazepam, SSRI's, NERI's, SNERI's, 5-HT1a agonists, and 5-HT2 antagonists, sedatives including but not limited to benzodiazepines, antipsychotics including but not limited to olanzapine, risperidone, haloperidol, clozapine, ziprazadone, loxapine, quetiapine and thioridazine, cognitive enhancers including but not limited to acetylcholinesterase inhibitors such as galantamine, rivastigmine, donepezil and NMDA antagonists such as mementine, neuroprotectants including but not limited to riluzole, topiramate, lamotrigine, dextromethorphine, mementine, and gastrointestinal motility enhancers and suppressants and dopamine receptor agonists including be not exclusive to pramipexole, ropinirole and pergolide; catechol-o-methyl transferase inhibitors including but not limited to entacapone and talcapone; dopa decarboxylase inhibitors including but not limited to carbidopa; alpha 2 adrenergic receptor agonists and antagonists such as clonidine and phentolamine; beta adrenergic antagonists such as propranolol and metoprolol; alpha 2 adrenergic agonists and antagonists and beta adrenergic antagonists.

The following examples are illustrative of the present invention, and are not to be construed as limiting the scope of the invention. Variations and equivalents of this example will be apparent to those of skill in the art in light of the present disclosure, the drawings and the claims herein.

All articles, books, patents and other publications referenced herein are hereby incorporated by reference in their entirety.

### EXAMPLES

### Example 1

### Osmotic Dosage Form

An osmopolymer hydrogel composition for use in the invention is prepared as follows: first 1274 g of pharmaceutically acceptable sodium carboxymethylcellulose comprising a 2,250,000 weight-average molecular weight, 600 g of sodium chloride, and 20 g ferric oxide are separately screened through a 40 mesh screen. Then, all the screened ingredients are mixed with 100 g of hydroxypropylmethylcellulose of 11,200 average-number molecular weight and 100 g of hydroxypropylcellulose of 30,000 average-number molecular weight to produce a homogenous blend. Next, 300 ml of denatured anhydrous alcohol is added slowly to the blend with continuous mixing for 5 minutes. Then, 1.6 g of butylated hydroxytoluene is added, followed by more blending, with 5 g of magnesium stearate added with 5 minutes of blending, to yield a homogenous blend. The freshly prepared granulation is passed through a 20 mesh screen and allowed to dry for 20 hours at 22.2° C. The final composition comprises 58.67 wt % sodium carboxymethylcellulose, 30 wt % sodium chloride, 1 wt % ferric oxide, 5 mg hydroxypropylmethylcellulose, 5 mg hydroxypropylcellulose, 0.08 wt % butylated hydroxytoluene, and 0.25 mg magnesium stearate.

The therapeutic 3-(2,3,5 -trichloro-phenyl)-pyrazine-2,6-diamine composition (17%) for a 25 mg dose and the osmopolymer hydrogel drug layer composition containing 200,000 MW polyethylene oxide (72.5%), sodium chloride (10%) and magnesium stearate (0.5%) are made into a bilayered tablet as follows: first, 147 mg of the drug layer is added to a punch die set and tamped. Then, 98 mg of the hydrogel composition is added and the two layers compressed under a pressure head of 1.0 ton (1000 kg) into a 11/32 inch (0.873 cm) diameter, contacting intimate bilayered tablet.

The bilayered tablet for example is manufactured into a dosage form as follows: first, a semipermeable wall-forming composition is prepared comprising 95 wt % cellulose acetate having a 39.8% acetyl content, and 5 wt % polyethylene glycol having a number-average molecular weight of 3350 by dissolving the ingredients in a cosolvent comprising acetone and water in 90:10 wt:wt composition to make a 4% solid solution. The wall-forming composition is sprayed onto and around the bilayered core to provide a 42 mg semipermeable wall.

Next, the semipermeable walled, bilayered tablet is laser drilled to provide a 25 mil (0.64 mm) orifice to contact the 3-(2,3,5-trichloro-phenyl)-pyrazine-2,6-diamine layer and the exterior of the dosage form. The residual solvent is removed by drying for 48 hours at 50 C and 50% relative humidity. Next, the dosage form is dried further for 1 hour at 50 C to remove excess moisture.

### Example 2

### Coated Bead Dosage Form

3-(2,3,5-trichloro-phenyl)-pyrazine-2,6-diamine was prepared as described in U.S. Pat. No. 6,803,464 and combined with the following components in the following amounts to obtain the Coated Bead Dosage form:

| | |
|---|---|
| First Bead Core | |
| 3-(2,3,5-trichloro-phenyl)-pyrazine-2,6-diamine | 518 grams |
| Microcrystalline cellulose | 300 grams |
| Hydroxypropylmethylcellulose | 50 grams |
| Sodium Monoglycerate | 80 grams |
| Tartaric Acid | 50 grams |
| Sodium Lauryl Sulfate | 2 grams |
| First Bead Coating: | |
| Eudragit RS 100 | 450 grams |
| Eudragit RL 100 | 450 grams |
| Propylene Glycol | 90 grams |
| Talc | 10 grams |
| Second Bead Core: | |
| 3-(2,3,5-trichloro-phenyl)-pyrazine-2,6-diamine | 518 grams |
| Microcrystalline cellulose | 300 grams |
| Hydroxypropylmethylcellulose | 50 grams |
| Sodium Monoglycerate | 80 grams |
| Tartaric Acid | 50 grams |
| Sodium Lauryl Sulfate | 2 grams |
| Second Bead Coating: | |
| Hydroxypropylmethylcellulose | 200 grams |
| Ethylcellulose | 700 grams |
| Polyethylene Glycol 400 | 100 grams |
| Third Bead Core: | |
| 3-(2,3,5-trichloro-phenyl)-pyrazine-2,6-diamine | 470 grams |
| Microcrystalline cellulose | 250 grams |
| Hydroxypropylmethylcellulose Phthalate | 100 grams |
| Sodium Monoglycerate | 75 grams |
| Tartaric Acid | 100 grams |
| Dioctyl Sodium Sulfosuccinate | 5 grams |
| Third Bead Coating: | |
| Cellulose Acetate Phthalate | 600 grams |
| Ethylcellulose | 250 grams |
| Polyethylene Glycol 400 | 150 grams |

For each of the beads, each of the materials is granulated and sieved and then admixed and agglomerated to form beads of reasonable size and robustness. The coating materials are then applied to the surfaces of the beads in a range of from about 1% (w/w) to about 25% (w/w). The beads are then combined to obtain the appropriate release profile (as shown, for example in FIG. 3) in the range of 5 to 25% of the First Bead, 15 to 70% of the Second Bead and 10 to 50% of the Third Bead.

### Example 3

### Matrix Dosage Form

3-(2,3,5-trichloro-phenyl)-pyrazine-2,6-diamine was prepared as described in U.S. Pat. No. 6,803,464 and combined with the following components in the following amounts:

| | | |
|---|---|---|
| 3-(2,3,5-trichloro-phenyl)-pyrazine-2,6-diamine | 450 | grams |
| Lactose (fast-flo) | 105 | grams |
| Microcrystalline cellulose | 714 | grams |
| Methocel E4M | 191.5 | grams |
| Methocel K100LV | 33.5 | |
| Magnesium Stearate | 6 | grams |

The components are then sieved using a Russel-SIV equipped with a 20 mesh (850 μm) or an equivalent sieve and mesh, and deposited into a stainless-steel blending container.

The 3 -(2,3,5-trichloro-phenyl)-pyrazine-2,6-diamine, lactose, microcrystalline cellulose and polymer are blended for 15 minutes using a suitable blender, such as a Matcon-Buls bin-type blender, a V-blender or equivalent. The magnesium stearate is then added to the mixture and blending continues for approximately 2 minutes.

The lubricated blend is then compressed using a suitable rotary tablet press, typically a Fette 2090 or equivalent to obtain a tablet with a release profile similar to that shown in FIG. 4.

### Example 4

### Dosage Form with Ascending Profile

The dosage form comprises 3 contacting layers of bioerodible poly(lactide-co-glycolide) with each layer containing an increased amount of 3-(2,3,5-trichloro-phenyl)-pyrazine-2,6-diamine. The layers are compressed into a laminated table-shaped arrangement with a single opened surface to expose the layer containing lo mg of 3-(2,3,5-trichloro-phenyl)-pyrazine-2,6-diamine with the remainder of the tablet surrounded with nonbioerodible copolymeric ethylene-vinyl acetate. The layers will bioerode in constant succession, corresponding to a constantly increasing dose of drug over time as shown, for example, in FIG. 5.

### Example 5

### Osmotic Dosage Form

Composition of the osmotic layer:

| Constituent | wt % |
|---|---|
| Polyethylene Oxide, NF, 303, 7000K, TG, | 64.30 |
| LEO | |
| Sodium Chloride, USP, Ph Eur, (Powder) | 30.00 |
| Povidone, USP, Ph Eur, (K29-32) | 5.00 |
| Magnesium Stearate, NF, Ph Eur, JP, | 0.25 |
| (Vegetable Sourced) | |
| BHT, FCC, Ph Eur, (Milled) | 0.05 |

Composition of the drug layer:

| Constituent | wt % |
|---|---|
| Polyethylene Oxide, NF, N750, 300K, TG | 89.8 |
| 3-(2,3,5-trichloro-phenyl)-pyrazine-2,6-diamine | 5.0 |
| Povidone | 5.00 |
| Magnesium Stearate, NF, Ph Eur, JP, (Vegetable Sourced) | 0.25 |

Composition of the semi-permeable membrane

| Constituent | wt % |
|---|---|
| Cellulose acetate | 99 |
| Polyethylene glycol | 1 |

The osmotic layer was prepared using a fluid bed granulator. The first two ingredients were placed in the chamber of the granulator. An aqueous solution of the third ingredient (povidone) was prepared separately. Heated air was introduced into the granulator to fluidize the mixture. The povidone solution was sprayed onto the dry ingredients and the resulting mixture was dried to a water content of less than 3%. The granulation was milled by forcing it through about a 20 mesh screen and then mixed with the magnesium stearate and butylated hydroxytoluene by dry blending.

The drug containing layer was prepared by mixing the first two ingredients in dry blender. The povidone was dissolved in ethanol and then sprayed onto the dry ingredients while being mixed at low speed. The resulting granulation was dried at ambient conditions overnight. The granulation was milled by forcing it through about a 20 mesh screen and then mixed with the magnesium stearate by dry blending.

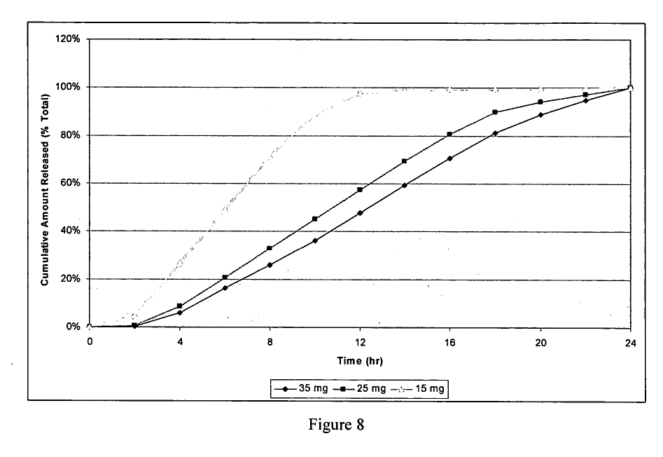

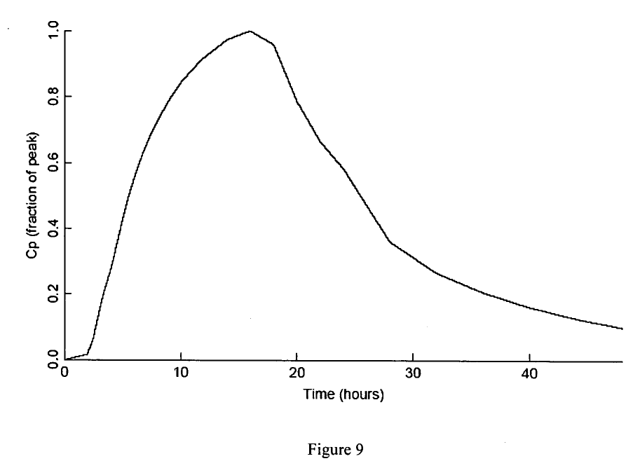

The two layers were compressed into tablets using a bi-layer press at a weight ratio of drug layer/osmotic layer of 1.2. The resulting tablets were then coated with the semi-permeable membrane in a pan coater to the desired weight. Two 0.64 mm holes were drilled in each coated core using a mechanical drill. The resulting systems were then dried for 2 days at 45° C. The release rate of the systems was controlled by the weight of the semi-permeable membrane that was applied to the tablets and are shown in FIG. 8. A simulation of the resulting plasma profile for a single dose for the 50 mg dosage form with 25 mg membrane is shown in FIG. 9.

### Example 6

### Coated Bead Dosage Form

3-(2,3,5-trichloro-phenyl)-pyrazine-2,6-diamine is prepared as described in U.S. Pat. No. 6,803,464 and combined with the following components in the following amounts to obtain the coated Bead Dosage form:

| | |
|---|---|
| First Bead Core | |
| 3-(2,3,5-trichloro-phenyl)-pyrazine-2,6-diamine | 82 grams |
| Sucrose beads | 820 grams |
| Ethylcellulose | 8 grams |
| Hydroxypropylcellulose | 10 grams |
| First Bead Seal Coating | |
| Hydroxypropylmethylcellulose | 16 grams |
| First Bead Enteric Coating: | |
| Hydroxypropylcellulose (HP-55) | 90 grams |
| Triethylcitrate | 9 grams |
| Second Bead Core: | |
| 3-(2,3,5-trichloro-phenyl)-pyrazine-2,6 diamine | 80 grams |
| Sucrose spheres | 800 grams |
| Hydroxypropylcellulose (HPC, LF) | 8 grams |
| Second Bead Seal Coating: | |
| Hydroxypropylmethylcellulose | 16 grams |

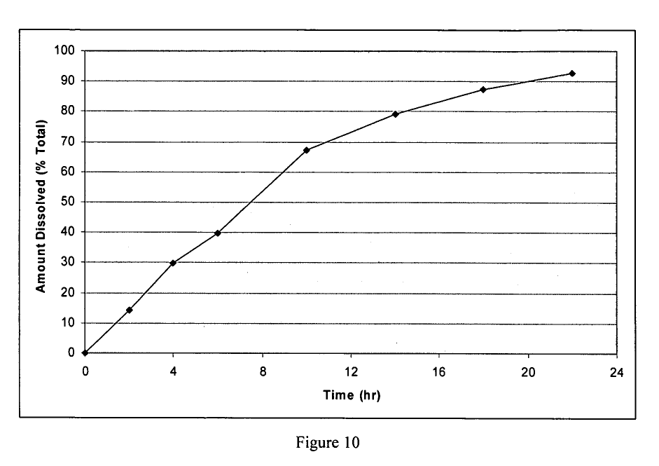

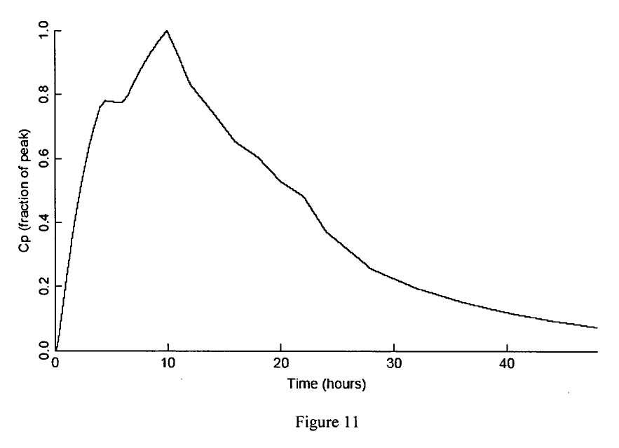

For each of the beads, the drug is dissolved in 25:75 acetone: 95% ethanol along with the binding ingredients (HC-55, EC10 or HPC,LF) and then spray coated onto the sucrose spheres. The drug coated spheres are then seal coated with hydroxypropylmethylcellulose (HPMC). The First Beads are coated with the enteric coating, dried and then sieved. The Second Beads are dried and sieved. The coating materials are then applied to the surfaces of the beads at 2 weight %. The beads are then combined to obtain the appropriate release profile (as shown, for example in FIG. 4) in the range of 85% of the First Bead, and 15% of the Second Bead. The second beads allow for an initial dose to be released followed by a sustained release dose facilitated by the first beads. Release rates for these beads are shown in FIG. 10 and the simulation for the plasma concentration corresponding to this release rate for the 50 mg dosage strength is shown in FIG. 11.

What is claimed is:

1. A sustained release dosage form comprising a substituted pyrazine derivative wherein the dosage form is effective to provide sustained release for at least about 8 hours.

2. The sustained release dosage form of claim 1 wherein the dosage form is effective to provide sustained release for at least about 12 hours.

3. A sustained release dosage form comprising a substituted pyrazine derivative wherein the dosage form is effective to provide a plasma profile at steady state wherein Cmax/Cmin is less than or equal to 3.

4. The sustained release dosage form of claim 1 wherein the dosage form is effective to provide a zero-order dissolution profile for a period of up to about 24 hours.

5. The sustained release dosage form of claim 4 wherein the dosage form is effective to provide a zero-order dissolution profile over a period of up to about 20 hours.

6. The sustained release dosage form of claim 1 wherein the dosage form is effective to provide a first-order dissolution rate for a period of up to about 24 hours.

7. The sustained release dosage form of claim 6 wherein the dosage form is effective to provide a first-order dissolution rate for a period of up to about 20 hours.

8. The sustained release dosage form of claim 1 wherein 2% and 50% of the dose is released following two hours after exposure to an aqueous environment.

9. The sustained release dosage form of claim 1 wherein 10% and 50% of the dose is released following four hours after exposure to an aqueous environment.

10. The sustained release dosage form of claim 1 wherein 20% and 80% of the dose is released following eight hours after exposure to an aqueous environment.

11. The dosage form of claim 1 comprising an osmotic dosage form.

12. The dosage form of claim 1 comprising a matrix dosage form.

13. The dosage form of claim 1 comprising a coated bead dosage form.

14. A method for treating a central nervous system disorder comprising administering to a human subject a sustained release dosage form of a substituted pyrazine derivative.

15. The method of claim 14 wherein the disorder is epilepsy.

16. A method for treating psychiatric disorder comprising administering to a human subject a sustained release dosage form comprising a therapeutically effective amount of a substituted pyrazine derivative.

17. The method of claim 14 wherein the disorder is selected from the group consisting of drug induced or naturally occurring dyskinesias, off-phase tachyphylaxis to L-DOPA treatment in Parkinson's, movement disorders, dementias or cognitive disorders associated with neurodegenerative disorders, fibromyalgia, acute and chronic pain, migraine and pseudobulbar affect.

18. The method of claim 16 wherein the disorder is selected from the group consisting of unipolar depression, bipolar depression, major depressive disorder, positive and negative symptoms of schizophrenia, post traumatic stress syndrome, acute mania, panic and psychotic reactions and attacks, conduct disorders, intermittent explosive or disruptive disorders, anxiety disorders, cognitive symptoms of schizophrenia, borderline personality disorder, attention deficit disorder, alcoholism and substance abuse.

19. The dosage form of claim 1 wherein the substituted pyrazine derivative is 3-(2,3,5-trichloro-phenyl)-pyrazine-2,6-diamine.

20. The method of claim 14 wherein the substituted pyrazine derivative is 3-(2,3,5-trichloro-phenyl)-pyrazine-2,6-diamine.

21. The method of claim 16 wherein the substituted pyrazine derivative is 3-(2,3,5-trichloro-phenyl)-pyrazine-2,6-diamine.

22. A dosage form for maintaining the therapeutic effect of 3-(2,3,5-trichloro-phenyl)-pyrazine-2,6-diamine comprising wherein the dosage form delivers the 3-(2,3,5-trichloro-phenyl)-pyrazine-2,6-diamine in a controlled and increasing dose over about 12 to 24 hours.

23. The dosage from of claims I wherein the substituted pyrazine derivative is selected from the group consisting of the M1, M2, Me, M4 and M5 metabolites of 3-(2,3,5-trichloro-phenyl)-pyrazine-2,6-diamine.
